(19) United States
(12) Patent Application Publication
(10) Pub. No.: US 2005/0092642 A1
(43) Pub. Date: May 5, 2005

(54) Package

(75) Inventor: Samuel Ombaku Nyambi, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/700,774

(22) Filed: Nov. 4, 2003

(51) Int. Cl. A61B 17/06

(52) U.S. Cl. 206/440

(57) ABSTRACT

The present invention relates to a package adapted to accommodate hygienic articles including a hemispherical shaped indention and an hemispherical extension. The package has a longitudinal axis and transverse axis. The package has a front surface and a back surface. Both the front surface and the back surface have at least one longitudinal side and first transverse side and a second transverse side. The package has a hemispherical shaped indention along the first transverse side and an equally sized and shaped hemispherical extension on the second traverse side.

### FIELD OF THE INVENTION

The present invention relates to a package adapted to accommodate hygienic articles comprising a hemispherical shaped indention and an hemispherical extension.

### BACKGROUND OF THE INVENTION

Absorbent articles need to be hygienically stored from the time of their manufacture until the article is used. Specifically, a need exists to hygienically store tampons, sanitary napkins, interlabial devices and pantiliners to prevent transferring unsanitary particles or moisture to the vaginal area.

The packaging for the commercially available FRESH 'N FIT® PADETTE® hygienic product is made from a coated paper sheet that is wrapped around the product and sealed with adhesive on the transverse ends and along the longitudinal edges and then crimped or knurled together. Other examples of packaging for absorbent article are shown in U.S. Pat. No. 4,743,245 entitled “Labial Sanitary Pad” that issued to F. O. Lassen, et al. on May 10, 1988, U.S. Pat. No. 3,062,371 entitled “Internally Sterile Composite Package” that issued to D. Patience on Nov. 6, 1962, U.S. Pat. No. 3,698,549 entitled “Packages for Small Articles” that issued to J. A. Glassman on Oct. 17, 1972, U.S. Pat. No. 3,135,262 entitled “Tampon” that, issued to W. Kobler, et al. on Jun. 2, 1964, and U.S. Pat. No. 5,180,059 entitled “Package of a Sanitary Tampon” that issued to S. Shimatani and K. Shimatani on Jan. 19, 1993.

Although the packages described in the prior art protect the enclosed article, one drawback is that these packages require the consumer to open the package in a tedious way that is not intuitive to the consumer sometimes causing the consumer to drop the absorbent article or cause the consumer's hand to touch the product. This is significant given the lack of hygiene in restrooms, the need to touch the doors of non-hygienic restrooms, and the necessity to touch themselves while inserting the device, which may result in a possible infection. One possible solution was devised individual package in combination with a hygienic device comprising a rupturable seal line adjacent to a permanent seal line comprising a pair of opening members situated on opposite sides of the rupturable seal line according to U.S. Pat. No. 6,478,763 issued to Simonsen, et al. on Nov. 12, 2002. While the package works for its intended purpose, the shape of the package generates a high percentage of scrap material which increases the cost of production.

Accordingly, it is an object of the present invention to provide package for absorbent articles that is intuitive and easy to open, providing hygienic product protection during its removal from a package and simultaneously during insertion or application, as well as, decreasing the material cost and the amount of scrap in production.

### SUMMARY OF THE INVENTION

The present invention relates to a package adapted to accommodate hygienic articles comprising a hemispherical shaped indention and an hemispherical extension. The package has a longitudinal axis and transverse axis. The package has a front surface and a back surface. Both the front surface and the back surface have at least one longitudinal side and first transverse side and a second transverse side. The package has a hemispherical shaped indention along the first transverse side and an equally sized and shaped hemispherical extension on the second traverse side.

### BRIEF DESCRIPTION OF THE DRAWINGS

FIG. 1 is a front view of a package according to the present invention.

FIG. 2 is a front view of the package of the present invention that is tom along the opening line.

FIG. 3 is a view of an absorbent articles housed with the pouch of the present invention.

### DETAILED DESCRIPTION OF THE INVENTION

The term “absorbent articles” refers to tampons, sanitary napkins, sanitary panties, interlabial devices, pantiliners, infant diapers, adult incontinence diapers, bandages, surgical swabs, and like that are used for the absorption of fluid and/or gas therefrom, to aid in wound healing, or for the delivery of active materials, such as medicaments, or moisture.

The term “interlabial pad,” refers to an absorbent article worn in the interlabial space for the absorption of fluid and/or gas therefrom, to aid in wound healing, or for the delivery of active materials, such as medicaments, or moisture. The interlabial pad comprises a liquid pervious topsheet, liquid impervious backsheet and an absorbent core diposed between the topsheet and the backsheet. Examples of such devices are described in U.S. Pat. No. 2,917,049 issued to Delaney on Dec. 15, 1959, U.S. Pat. No. 3,420,235 issued to Harmon on Jan. 7, 1969, U.S. Pat. No. 4,595,392 issued to Johnson, et al. on Jun. 17, 1986, and U.S. Pat. No. 5,484,429 issued to Vukos, et al. on Jan. 16, 1996. A commercially available interlabial device is the INSYNC Miniform interlabial pad which is marketed by A-Fem of Portland, Oreg. and described in U.S. Pat. Nos. 3,983,873 and 4,175,561 issued to Hirschman on Oct. 5, 1976 and Nov. 27, 1979, respectively.

The term “hygienic articles” as used herein includes absorbent articles, wet wipes and the like used for maintenance of a bodily hygiene.

As used herein the term “is” is the common dictionary definition and in that used in common parlance.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

As used herein, the terms “pantiliner,” and “sanitary napkin”, refers to absorbent articles worn external about the pudenal region for the absorption of fluid and/or gas therefrom, to aid in wound healing, or for the delivery of active materials, such as medicaments, or moisture. Sanitary napkins typically comprise a liquid pervious topsheet, liquid impervious backsheet and an absorbent core diposed between the topsheet and the backsheet. The sanitary napkin, as well as each layer or component thereof can be described as having a “body facing” surface and a “garment facing” surface. As can be readily understood by considering the ultimate use for sanitary napkins, the body facing surfaces are the surfaces of the layers or components that are oriented closer to the body of the user when in use, and the garment facing surfaces are the surfaces that are oriented closer to the undergarment of the user when in use. Therefore, for example, the topsheet has a body facing surface and a garment facing surface that is the surface that can be adhered to the underlying first absorbent layer. The garment facing surface of the fluid impermeable backsheet of a pantiliner or sanitary napkin, for example, is oriented closest to and may contact the wearer's panties in use via adhesive attachment means, if used. Pantiliners and sanitary napkin may have side extensions commonly referred to as “wings,” designed to wrap the sides of the crotch region of the panties of the user of sanitary napkin that may be extension of the topsheet and/or the backsheet. Such devices are disclosed in U.S. Pat. No. 4,463,045 issued to Ahr et al., U.S. Pat. No. 4,556,146 issued to Swanson et al., U.S. Pat. No. 4,950,264 issued to Osborn III, et al. and U.S. Pat. No. 4,687,478 issued to Van Tillburg.

As used herein, the term, “substantially parallel” refers to the orientation of a first line to second line or lines. Substantially parallel means that the first line may be 85 degrees to 95 degrees from the second line.

As used herein the term “tampon,” refers to any type of absorbent structure that is inserted into the vaginal canal or other body cavities for the absorption of fluid and/or gas therefrom, to aid in wound healing, or for the delivery of active materials, such as medicaments, or moisture. Tampon pledget may be constructed from a wide variety of liquid-absorbing materials commonly used in absorbent articles. Such materials include but are not limited to rayon (such as GALAXY Rayon SARILLE L rayon both available from Acordis Fibers Ltd., of Hollywall, England), cotton, folded tissues, woven materials, nonwoven webs, synthetic and/or natural fibers or sheeting, comminuted wood pulp which is generally referred to as airfelt, or combinations of these materials. Tampons invention may optionally comprise an overwrap, secondary absorbent or skirt comprising material such as, rayon, cotton, bicomponent fibers, polyethylene, polypropylene, other suitable natural or synthetic fibers known in the art, and mixtures thereof. . Tampons are typically compressed and/or shaped such that it assumes a general shape and size, which is vaginally insertable, absent external forces. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as rectangular, triangular, trapezoidal, semi-circular, hourglass, serpentine, or other suitable shapes. Tampons have an insertion end, withdrawal end, a length, a width, a longitudinal axis, a radial axis and an outer surface. The tampon's length can be measured from the insertion end to the withdrawal end along the longitudinal axis. A typical compressed tampon for human use is 30-60 mm in length. A tampon may be straight or non-linear in shape, such as curved along the longitudinal axis. A typical compressed tampon is 8-20 mm wide. The width of a tampon, unless otherwise stated in the specification, corresponds to the length across the largest cylindrical cross-section, along the length of the tampon. Tampons may comprise withdrawal members comprised of any suitable material known in the prior art and include cotton and rayon. Tampons may be digital tampon refers to a tampon which is intended to be inserted into the vaginal canal with the user's finger and without the aid of an applicator and are typically visible to the consumer prior to use rather than being housed in an applicator. Alternatively, the insertion may be aided through the use of any applicator adapted from the prior art including a typical “tube and plunger” type arrangement made of plastic, paper, or other suitable material. Such absorbent articles are disclosed in U.S. Pat. No. 5,087,239 issued to Beastall et al., U.S. Pat. No. 5,279,541 issued to Frayman et al.; U.S. Pat. Nos. 6,258,075 and 6,599,279 both issued to Taylor, et al., U.S. patent application Ser. No. 10/150,050, filed Mar. 18, 2002, entitled “Substantially Serpentine Shaped Tampon,” to Randall, et al. and currently pending and commonly assigned, U.S. patent application Ser. No. 10/150,055, filed Mar. 18, 2002, entitled “Shaped Tampon,” to Kollowitz, et al.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

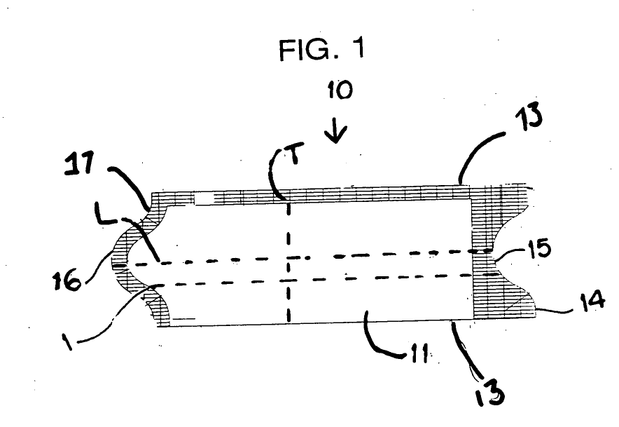

The present invention, as shown in FIG. 1, provides an easy opening package 10 that is cut to a desired shape and sealed, such as by thermo-mechanical methods known in the art, to form packages 10. The packages 10 of the present are the same thickness as other conventional packages 10, between 20 μm and 50 μm um, or about 32.0 μm to about 42 μm. The package 10 may be any shape known in the art and any size that will accommodate its contents. If the package is intended to house a digital tampon the package may be from about 55 mm to about 60 mm in width and from about 80 mm to about 83 mm in length. The walls of the package can be of any desired thickness, commensurate with the intended use. Preferably, the walls are flexible and fluid-impermeable. Typically, the walls of package 2 have a thickness of from about 0.0127 mm (0.5 mil) to about 0.127 mm (5.0 mils). The package 2 may be made from plastic films that may be at thermoplastic film, nonwoven materials, collagen films, paper tissues, or laminates of tissue and a film, nonwoven material and a film, or any of the foregoing types of material with a coating thereon.

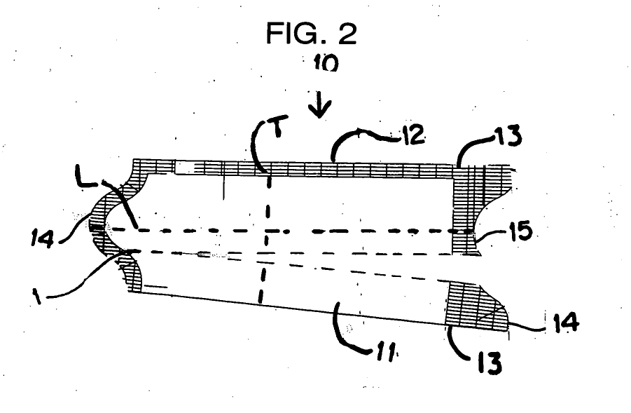

The package 10, as shown in FIG. 1 and FIG. 2, has a longitudinal axis L and transverse axis T. The package has a front surface 11 and a back surface 12. Both the front surface 11 and the back surface 12 have at least one longitudinal sides 13 and first transverse side 14 and a second transverse side 17. The package 10 in FIG. 1 and FIG. 2 has a hemispherical shaped indention 15 along the first transverse side 14 and an equally sized and shaped hemispherical extension 16 on the second traverse side 17. This configuration of the package reduces the amount of scrap material in the cutting process. As well, it is believe that the configuration of the package allows for the consumer to intuitively know where the proper opening is without use of graphics. In FIG. 2 the package 10 is opened along the intended opening axis 1, which may be substantially parallel to the longitudinal axis L.

The present invention, as shown in FIG. 1 and FIG. 2, provides an easy opening package 10 that is cut to a desired shape and sealed, such as by thermo-mechanical methods known in the art, to form packages 10. The seal strength of the package 10 of the present invention is typically greater that the tensile strength of the film laminate comprising the package 10. The package 10 of the present are the same thickness as other conventional packages 10, between 20 μm and 50 μm, or about 32 μm to about 42 μm thick. The packages 10 may be any shape known in the art and any size that will accommodate its contents. If the package is intended to house a digital tampon the package may be from about 55 mm to about 60 mm in width and from about 80 mm to about 83 mm in length.

A suitable method for commercial production of the wrapper is to roll feed in flow wrap-type system. First, the material comprising the package 10 is folded the wrapper on its longitudinal axis L. Next, the material comprising the package 10 is die cut the hemispherical indention 15 on the first transverse side 14 and hemispherical extension 16 on the second transverse side 17. Then, the package 10 is sealed on the first transverse side 14 and the longitudinal side 13. Next, the hygienic article is inserted through the second transverse side 17 substantial parallel to the longitudinal side 13; seal the second transverse side 17. In embodiments of the package for dispensing a digital tampon the seal of the first transverse side 14 and the second transverse side 17 are from about 2 mm to about 4 mm wide and the seal of the longitudinal side 13 is from about 10 mm to 13 mm wide. The configuration of the package 10 is such that when the hemispherical indention 15 of a first package is die cut the hemispherical extension 16 on a second package 10 is cut and nested within the hemispherical indention 15 of the first package. This configuration of the package 10 reduces the amount of scrap material in the cutting process. Thus, the amount of material wasted in cutting this configuration may be negligible, including between from about 0% to about 5% of the material used to produce the package 10.

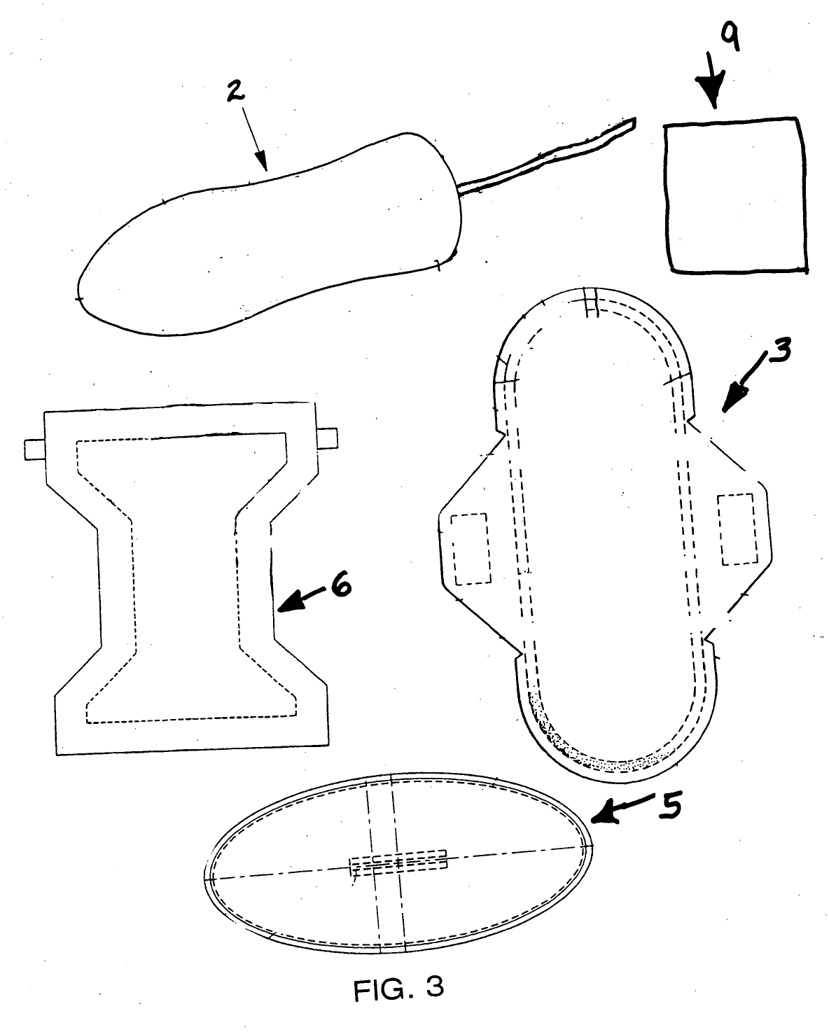

FIG. 3 shows absorbent articles that may be stored in the package of the present invention, including a tampon 2, sanitary napkin 3, interlabial device 5, and diaper 6 and a wipe 9.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the scope of the invention. It is therefore intended to cover in the appended claims all such changes and modifications that are within the scope of this invention.

1. The package adapted to accommodate hygienic article comprising:
a. a front surface and a back surface;
b. said front surface and said back surface comprising a longitudinal axis; a transverse axis; at least one longitudinal side; a first transverse side and a second transverse side;
c. said first transverse side comprises a hemispherical shaped indention; and said second tranvservse side comprises a hemispherical extension.

2. The package according to claim 1 wherein said hemispherical extension and said hemispherical indention are equally sized and shaped.

3. The package according to claim 1 further comprising an intended opening axis.

4. The package according to claim 4 wherein said intended opening axis is substantially parallel to said longitudinal axis.

5. The package according to claim 1 wherein said package is comprised of thermoplastic film.
